(19) United States
(12) Patent Application Publication
(10) Pub. No.: US 2007/0299246 A1
(43) Pub. Date: Dec. 27, 2007

(54) IN VITRO PROTEIN EXPRESSION PROCESS COMPRISING Ckappa FUSION MOLECULES

(76) Inventors: Michael John Taussig, Cambridgeshire (GB); Mingyue He, Cambridgeshire (GB)

Correspondence Address: HOWREY LLP, C/O IP DOCKETING DEPARTMENT, 2941 FAIRVIEW PARK DRIVE, SUITE 200, FALLS CHURCH, VA, 22042-2924, US

(21) Appl. No.: 11/766,050

(22) Filed: Jun. 20, 2007

Related U.S. Application Data
(60) Provisional application No. 60/805,397, filed on Jun. 21, 2006.

(51) Int. Cl. C07K 1/00

(52) U.S. Cl. 530/338

(57) ABSTRACT

The invention relates to an in vitro protein expression process comprising preparation of a nucleic acid molecule which comprises a fusion of a gene encoding a target protein and a gene encoding an immunoglobulin κ light chain constant domain (Cκ) followed by cell-free protein expression. The invention also relates to proteins expressed by said process and to a composition and kit for performing said in vitro protein expression process.

This application claims priority to U.S. provisional application No. 60/805,397, filed Jun. 21, 2006. The content of which is incorporated herein by reference in its entirety.

### TECHNICAL FIELD OF THE INVENTION

The invention relates to an in vitro protein expression process comprising preparation of a nucleic acid molecule which comprises a fusion of a gene encoding a target protein and a gene encoding an immunoglobulin κ light chain constant domain (Cκ) followed by cell-free protein expression. The invention also relates to proteins expressed by said process and to a composition and kit for performing said in vitro protein expression process.

### BACKGROUND OF THE INVENTION

Protein production in heterologous systems is a major challenge in many areas of biological research and biopharmaceutical development. Cell-free protein synthesis is becoming a widely used alternative to cell-based methods for rapid and parallel production of proteins, providing a rapid route to the translation of genetic information into folded proteins. As in vitro methods, cell free expression systems allow proteins to be expressed and modified during translation under defined conditions that living cells may be incapable of reproducing. As well as their application to protein production for structural and functional studies (Spirin, A. (2004) TIB 22, 538-545), a number of significant protein selection and display technologies, including ribosome display, mRNA display and in situ protein arrays, also make use of cell-free protein expression systems (He, M. & Taussig, M J. (1997) Nucleic Acids Res. 25, 5132-5134; Hanes, J. & Pluckthun, A. (1997) Proc. Natl. Acad. Sci. USA. 94, 4937-4942; He, M. & Taussig, M. J. (2001) Nucleic Acid. Res. 29, e73).

A number of cell-free protein expression systems are available, such as rabbit reticulocyte, E. coli S30, and wheat-germ lysates, mammalian cells (Mikami, S. et al., (2006) Protein Expr. Purif. 46, 348-357) and the artificially assembled PURE system (Shimizu, Y. et al., (2001) Nat. Biotechnol. 119, 781-755). Efforts have been made to improve protein yield by identifying key factors affecting in vitro transcription and translation and developing modified protocols. They include the preparation of cell-free extracts using genetically engineered bacterial strains, optimisation of extraction of the cell lysate, supplies of various energy resources or amino acid concentration, defining the composition of the system using isolated components, and the use of dialysis, continuous-flow, continuous exchange, hollow fiber systems, and bilayer and the film-surface (Spirin, A. (2004) TIB 22, 538-545; Calhoun, K. & Swartz, J. R. (2005) Biotechnol. Prog. 21, 1146-1153; Sawasaki, T. et al., (2002) FEBS Lett. 514, 102-105). Despite these developments, some proteins are still only weakly expressed in cell-free systems. There is thus a great need to enhance cell-free protein expression.

### SUMMARY OF THE INVENTION

According to a first aspect of the invention there is provided an in vitro process of expressing a target protein which comprises the steps of:
(a) preparing a nucleic acid construct comprising both a gene encoding said target protein and a gene encoding an immunoglobulin κ light chain constant domain (Cκ); and
(b) subjecting the construct prepared in step (a) to protein expression in a cell-free protein expression system.

According to a second aspect of the invention there is provided a target protein-Cκ domain fusion protein obtainable by a process as hereinbefore defined.

According to a further aspect of the invention there is provided a protein obtainable by a process as hereinbefore defined.

According to a further aspect of the invention there is provided a composition for in vitro expression of a target protein, said composition comprising:
(a) a nucleic acid construct comprising both a gene encoding said target protein and a gene encoding an immunoglobulin κ light chain constant domain (Cκ); and
(b) a cell-free protein expression system.

According to a further aspect of the invention there is provided a kit for in vitro protein expression of a target protein which comprises:
(a) a nucleic acid construct comprising a gene encoding an immunoglobulin κ light chain constant domain (Cκ); and
(b) a cell-free protein expression system.

According to a further aspect of the invention there is provided a nucleic acid molecule comprising both a gene encoding a non-immunoglobulin protein and a gene encoding an immunoglobulin κ light chain constant domain (Cκ).

### BRIEF DESCRIPTION OF THE FIGURES

FIG. 1 shows a representation of a typical nucleic acid construct in accordance with one embodiment of the invention.

FIG. 2 shows the effect of Cκ fusion upon human single chain (sc) antibody expression. FIG. 2A shows anti-progesterone scFv fragment. FIG. 2B shows anti-CEA scFv fragment. Lane 1 represents construct without Cκ domain, and Lane 2 represents construct with Cκ domain.

FIG. 3 shows the effect of Cκ fusion upon the expression of Rab22b (a GTP binding protein). FIG. 3A was detected by anti-His antibody. FIG. 3B was detected by anti-Cκ antibody. Lane 1 represents construct without Cκ domain, and Lane 2 represents construct with Cκ domain.

FIG. 4 shows the effect of Cκ fusion upon FK506 binding protein expression. FIG. 4A was detected by anti-His antibody. FIG. 4B was detected by anti-Cκ antibody. Lane 1 represents construct without Cκ domain, and Lane 2 represents construct with Cκ domain.

FIG. 5 shows a representation of the nucleic acid constructs with (FIG. 5B) and without Cκ (FIG. 5A) which were used in the transcription factor analysis.

FIG. 6 shows the effect of Cκ fusion on expression of human transcription factors. FIG. 6A shows four constructs without Cκ fusion, and FIG. 6B shows four constructs with Cκ fusion.

### DETAILED DESCRIPTION OF THE INVENTION

The process of the invention provides the advantage of significantly enhancing protein expression when incorporated into an in vitro protein expression system (e.g. a cell-free system comprising components for transcription and translation) which consequently allows the production of proteins in a quantity which were previously only scarcely produced. Cκ has been described as a spacer for ribosome display of antibody fragments (He, M. and Taussig, M. J. (1997) supra). Subsequently, Chen, S. S. et al. (2004) FASEB J. 18, pC173, abs no. 73.10 described the use of a Cκ fusion protein for ribosome display of GFP, however, the use of Cκ constructs presented herein provides a new approach to enhance in vitro protein production. In vivo fusion of a Cκ domain to immunoglobulin and closely related proteins (e.g. single chain antibody fragments and T-cell receptor protein) is known (Maynard, J. A. et al. (2002) Nature Biotechnol. 20, 597-601; Maynard, J. A. et al. (2005) J. Immunol. Methods. 306, 51-67). The Cκ domain has also been used to make fusions with other non-immunoglobulin proteins for in vivo expression (Caswell, R. et al. (1993) Biotechnol. Techniques 7(4), 307-312; WO 2005/087810 (Zymogenetics Inc.); U.S. Pat. No. 6,146,631 (Better et al.); WO 01/46232 (Zymogenetics Inc.) and EP 0 505908 (F. Hoffmann La Roche AG)), however, the results shown in at least Caswell et al. demonstrated that the resultant fusion protein could not be detected. By contrast, data presented herein surprisingly demonstrates that protein expression is significantly enhanced in a cell-free protein expression system.

In one embodiment, the protein expression system comprises an in vitro protein expression system (e.g. a cell-free system) comprising components for transcription and translation. In a further embodiment, the cell-free system is a cell-free lysate selected from a prokaryotic or eukaryotic system, such as E. coli, rabbit reticulocyte, wheatgerm lysates, mammalian cells ((Mikami, S. et al., (2006) supra) or an artificially constructed system (e.g. the PURE system (Shimizu, Y. et al., (2001) supra) which enables protein synthesis in vitro. In a further embodiment, the cell-free system is a bacterial cell-free system such as E. coli (e.g. a coupled E. coli S30 cell-free system). In the embodiment wherein the nucleic acid construct comprises an mRNA construct, the cell-free system used for protein expression in step (b) is suitably an uncoupled cell-free system for translation.

It will be appreciated that the cell-free protein expression system will be capable of full expression of the target protein, for example, the process will result in a solubilised, expressed protein.

References to “nucleic acid” refer to any nucleic acid moiety capable of in vitro protein synthesis when exposed to an in vitro protein expression system (e.g. a cell-free system comprising components for transcription and translation). In one embodiment, the nucleic acid moiety comprises genomic DNA, cloned DNA fragments, plasmid DNA, cDNA libraries, PCR products, synthetic oligonucleotides or mRNA. The nucleic acid constructs for in vitro transcription/translation may be obtained by PCR (polymerase chain reaction) or RT (reverse transcription)-PCR amplification, using primers designed on any known DNA sequences, such as those from databases and genome projects.

In one embodiment, the nucleic acid molecule comprises a PCR product.

References to “target protein” refer to any protein required to be expressed and/or purified and/or characterised. Data is presented herein to demonstrate the applicability of the invention for enhancing the level of expression of a variety of proteins and therefore the definition of target protein is intended to be defined broadly. In one embodiment, the target protein is an immunoglobulin protein. In an alternative embodiment, the target protein is a non-immunoglobulin protein. In a further embodiment, the non-immunoglobulin protein is a binding protein (e.g. a GTP binding protein, such as Rab22b or an FK506 binding protein, such as FKBP2). In an alternative embodiment, the non-immunoglobulin protein is a transcription factor (e.g. human transcription factor) such as ERG, E2F-1, SMAD3 or TCF7L2. In a further embodiment, the human transcription factor is ERG, E2F-1 or SMAD3.

In one embodiment, the immunoglobulin κ light chain constant domain (Cκ) is a human immunoglobulin κ light chain constant domain (Cκ).

In one embodiment, the gene encoding the Cκ domain is fused, suitably with a peptide linker, to the gene encoding the target protein. It will be appreciated that the Cκ domain may be present at either the N-terminus or C-terminus of the gene encoding the target protein. In one embodiment, the gene encoding the Cκ domain is present at the C-terminus of the gene encoding the target protein.

Alternative tags, e.g. the chloramphenicol acetyl transferase (CAT) sequence, have been used to engineer proteins to increase their expression level in cell-free systems, however, these have generally been N-terminal fusions (Son, J. M. et al. (2006) Anal. Biochem. 351, 187-192; Shaki-Loewenstein, S. et al. (2005) J. Immunol. Meth. 303, 19-39). The resultant construct therefore increases translation initiation, and consequently also production of the overall protein. Contrary to these results, we have surprisingly shown that fusion of Cκ to the C-terminus of a target protein significantly enhances in vitro expression of the target protein.

The nucleic acid molecule may additionally comprise one or more of the following: a promoter, a transcriptional and translational regulatory sequence, an untranslated leader sequence, a sequence encoding a cleavage site, a recombination site, a transcriptional terminator or a ribosome entry site. The nucleic acid molecule may further comprise a plurality of cistrons (or open reading frames) or a sequence encoding a reporter protein whose abundance may be quantitated and can provide an accurate measure of expressed protein.

In one embodiment of the invention the nucleic acid molecule comprises one or more promoter (e.g. a T7 promoter), enhancer (e.g. a gene10 enhancer) and a ribosome binding site or translation initiation sequence (e.g. a Shine Dalgarno (SD) sequence for prokaryotic systems or kozak sequence for eukaryotic systems). Such sequences are either commercially available and may be purchased, for example, from Roche or may be prepared in accordance with standard methodology.

In one embodiment the nucleic acid molecule comprises one or more transcriptional and translational terminators present at the 3′ end of the molecule.

In order to enhance the efficiency of isolation of the resultant expressed target protein, the nucleic acid molecule may additionally comprise a gene encoding an immobilisation tag configured to attach (e.g. covalently or non-covalently) to a protein immobilisation agent.

In one embodiment, the immobilisation tag is a polyhistidine sequence, such as one or more hexahistidine and said protein immobilisation agent is a chelating agent such as Ni-NTA. In a further embodiment, said immobilisation tag is a peptide, domain or protein and said protein immobilisation agent is an antibody specific to said tag.

It will be appreciated that preparation of the nucleic acid construct in step (a) may be performed in accordance with standard molecular biology techniques known to those skilled in the art, for example, those described in He, M. & Taussig, M. J. (2001) Nucleic Acid Res. 29, e73, the nucleic acid construct protocols of which are herein incorporated by reference.

In one embodiment, the process of the invention additionally comprises the step of:
(c) isolating the expressed target protein from the protein expression system.

The presence of the Cκ domain within the resultant expressed fusion protein of target protein-Cκ domain significantly simplifies isolation of the fusion protein from the protein expression system. For example, the isolation step (c) may be performed by known techniques such as affinity chromatography and the like, which would involve an agent capable of recognising and binding to the Cκ domain. Thus, the presence of the Cκ domain not only provides the significant advantage of enhancing protein expression but also synergistically provides a useful immobilisation tag in which the resultant protein may be isolated without the need for incorporation of additional immobilisation tags. In one embodiment, the isolation step (c) involves affinity chromatography or the like with agents having affinity for the Cκ domain (e.g. antibodies or Protein L).

It will be appreciated that the isolation step (c) may be replaced with a detection step wherein detection techniques, such as immunodetection, Western blotting and the like, may be employed to detect the presence of the expressed target protein.

In one embodiment, the process of the invention additionally comprises the step of:
(d) cleaving the Cκ domain from the expressed target protein.

It will be appreciated that cleavage of the Cκ domain may be required in order to perform structural and functional studies of the target protein. It will also be appreciated that the cleavage step (d) may be performed either before or after isolation step (c). In the embodiment wherein the protein expression system is an E. coli cell-free system, cleavage may typically be performed by in situ specific cleavage at an engineered protease site in the E. coli cell-free translation mixture. Such cleavage may be performed by standard procedures known to those skilled in the art, for example, those described in Son, J. M. et al. (2006) Anal. Biochem. 351, 187-192, the tag cleavage protocols of which are herein incorporated by reference.

An advantage of using an in vitro protein expression system (e.g. a cell-free system) is that they provide an environment in which the conditions of protein expression can be adjusted and controlled through addition of exogenous biomolecules or molecules. This makes it possible to generate modified proteins, such as those with co- or post-translational modifications, non-natural or chemically modified amino acids (such as fluorescent groups).

Thus, in one embodiment of the invention, the protein expression system contains one or more additional agents. In an alternative embodiment, the nucleic acid molecule may comprise a gene encoding one or more additional agents (e.g. gene encoded products such as polypeptides or RNA molecules).

In one embodiment, the additional agents interact with the expressed fusion or target protein or encode additional agents capable of interacting with the fusion or target protein (e.g. nucleic acids capable of being transcribed and/or translated into a protein binding partner by the protein expression system).

In a further embodiment, the additional agents are biomolecules or molecules required to produce modifications such as co- or post-translational modifications, non-natural or chemically modified amino acids (such as fluorescent groups or biotin). In a yet further embodiment, the additional agents are reporter proteins such as an enzyme (e.g. β-galactosidase, chloramphenicol acetyl transferase, β-glucuronidase or the like) or a fluorescent protein (e.g. green fluorescent protein (GFP), red fluorescent protein, luciferase or the like). The additional agents are suitably added into the cell-free lysate, such that the resultant expressed proteins are modified during translation or after immobilisation and may allow the rapid detection of such proteins. In one embodiment, the additional agent comprises one or more protein folding promoting agents. These agents have the advantage of ensuring that the expressed protein is correctly folded.

It is envisaged that the kit of the invention will enable the user to incorporate the gene encoding the target protein into the nucleic acid construct of component (a) and then simply express the target protein in accordance with the process as hereinbefore defined.

In one embodiment, the kit additionally comprises instructions to use said kit in accordance with the process as hereinbefore defined.

It will be appreciated that the kit or composition for in vitro protein expression of a target protein may additionally comprise any other component or feature hereinbefore described with reference to the process of the invention.

The invention will now be described, by way of example only, with reference to the accompanying examples:

### EXAMPLES

1. Materials Used

Primers

| | | | |
|---|---|---|---|
| (1) | RTST7/B: | 5′-GATCTCGATCCCGCG-3′ | (SEQ ID NO: 1) |
| (2) | PET7/F: | 5′-CATGGTGGATATCTCCTTC | (SEQ ID NO: 2) |
| | | TTAAAG-3′ | |
| (3) | Linker- | 5′-GCTCTAGAGGCGGTGGC- | (SEQ ID NO: 3) |
| | tag/B: | 3′ | |
| (4) | Tterm/F: | 5′-TCCGGATATAGTTCCTC | (SEQ ID NO: 4) |
| | | C-3′ | |
| (5) | HuC4/B: | 5′-GTGGCTGCACCATCTGTC | (SEQ ID NO: 5) |
| | | T-3′ | |
| (6) | RzpdCk/F: | 5′-AGATGGTGCAGCCACAGTT | (SEQ ID NO: 6) |
| | | TTGTACAAGAAAGCTGGG-3′ | |
| (7) | PErzpd/B: | 5′-CTTAAGAAGGAGATATCCA | (SEQ ID NO: 7) |
| | | CCATGCTCGAATCAACAAGTTT | |
| | | GTAC-3′ | |
| (8) | Rzpd-L/F: | 5′-GCCACCGCCTCTAGAGCGT | (SEQ ID NO: 8) |
| | | TTGTACAAGAAAGCTGG-3′ | |

Molecular Biology Reagents and Cell-Free System

Nucleotides, agarose, PCR Gel Extraction Kit and HRP-linked mouse anti-His antibody were obtained from Sigma, UK; Taq DNA polymerase was obtained from Qiagen, UK; HRP-linked anti-human K antibody was obtained from the Binding Site, UK; NuPAGE Bis-Tris gels were obtained from Invitrogen, CA, USA; PVDF Immobilon-P membranes were obtained from Millipore; Western Blot detection SuperSignal Kit was obtained from Pierce, UK; and coupled E. coli S30 cell-free expression system was obtained from Roche, UK. The rab22b and FKBP2 clones were obtained from Dr. Bernhard Korn.

2. Construction of PCR Fragments

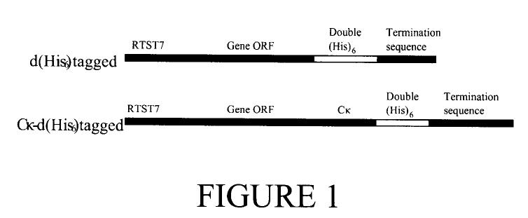

The general PCR constructs used for cell-free protein synthesis are shown in FIG. 1. The 5′ end contains a T7 promoter, a gene10 enhancer and SD sequence (Roche kit) for efficient transcription and translation. The ORF of the gene of interest was placed after the initiation codon ATG, followed by fusion in frame to the following in order: a flexible peptide linker, a double-(His)₆ tag and two consecutive stop codons (TAATAA) (He, M. & Taussig, M. J. (2001) Nucleic Acid. Res. 29, e73). When human Cκ was included, it was placed downstream of the gene ORF and before the peptide linker. A transcription termination region was included at the 3′ end of the constructs.

3. PCR Generation of Individual Domains

The standard PCR mixture consisted of 5 μl 10×PCR buffer, 10 μl 5×Q, 4 μl dNTPs mix containing 2.5 mM of each, 1.5 μl of forward and backward primers (16 μM each), 1 U Taq DNA polymerase, 1-10 ng template DNA and water to a final volume of 50 μl.

(a) RTST7 domain, comprising T7 promoter, gene10 enhancer and SD sequence, was created using primers RTST7/B and PET7/F from a plasmid template used as a control in the cell free system (Roche, UK).

(b) Double (His)₆ tag domain, comprising a flexible peptide linker, two hexahistidine sequences, separated by an 11-amino acid spacer sequence, and two consecutive stop codons (TAATAA), was generated using primers Linker-tag/B and Tterm/F on the plasmid template pTA-His (He, M. & Taussig, M. J. (2001) Nucleic Acid. Res. 29, e73).

(c) Cκ-(His)₆ tag domain was produced using primers HuC4/B and Tterm/F on a plasmid template, which encodes the Cκ domain with the double-(His)₆ tag fused at the C-terminus.

(d) ORFs of genes to be expressed were amplified using their corresponding plasmids (RZPD, Germany) as templates and individually designed primers. For generation of constructs without Cκ, primer PErzpd/B and Rzpd-L/F were used, while PErzpd/B and RzpdCκ/F were used for constructs with Cκ.

4. Assembly PCR

The ORF of the gene of interest and the appropriate domain fragments were assembled by mixing in equimolar ratios (total DNA 50-100 ng) after elution from agarose gel (1%) and adding into a PCR solution containing 2.5 μl 10×PCR buffer, 1 μl dNTPs mix containing 2.5 mM of each, 1 U Taq DNA polymerase and water to a final volume of 25 μl, and thermal cycling for eight cycles (94° C. for 30 s, 54° C. for 1 min and 72° C. for 1 min). For constructs without Cκ the fragments assembled were the RTST7 domain, gene ORF and double(His)₆ tag domain, while for the constructs with Cκ they were the RTST7 domain, gene ORF and Cκ-(His)₆ tag domain.

5. Amplification of PCR Constructs

Assembled constructs were amplified by transferring 2 μl to a second PCR mixture in a final volume of 50 μl (as above) for a further 30 cycles using primers RTST7/B and T-term/F. Thermal cycling for 30 cycles (94° C. for 30 s, 54° C. for 1 min and 72° C. for 1 min, finally, 72° C. for 8 min). The final PCR construct was analysed by agarose (1%) gel electrophoresis to determine quality and concentration by comparison with a known DNA marker. The PCR products may be used for cell-free expression with or without further purification.

6. Cell-Free Protein Expression

Proteins were expressed from PCR constructs using the coupled E. coli S30 system, incubated at 30° C. for 4 hours. A standard reaction comprised 12 μl E. coli S30 lysate, 12 μl amino acids, 10 μl reaction mix, 5 μl reconstitution buffer, 1 μl methionine and 100-500 ng PCR DNA, made to 50 μl with water.

7. Detection of Proteins by Western Blotting

Protein expressed in the E. coli S30 lysate were mixed with an equal volume of 2×SDS buffer (100 mM Tris, pH 8.0, 5% SDS, 0.2% bromophenol blue, 20% glycerol), heated to 90° C. for 5 minutes, loaded onto a 10% NuPAGE Bis-Tris gel and run at 200V. The separated proteins were transferred to a PVDF membrane by electroblotting for 2 hours at 80 mA. The membrane was blocked in 1% bovine serum albumin (BSA) in phosphate-buffered saline (PBS) for 1 hour, then incubated with either HRP-linked mouse anti-His antibody (diluted 1:4000 in PBS/BSA) or HRP-linked mouse anti-κ antibody (1:500 in PBS/BSA) for 1 hour. The membrane was developed using the SuperSignal kit (PIERCE, UK) as per the manufacturer's instructions.

### Example 1

### Effect of Cκ Fusion on Human Single Chain (sc) Antibody Expression

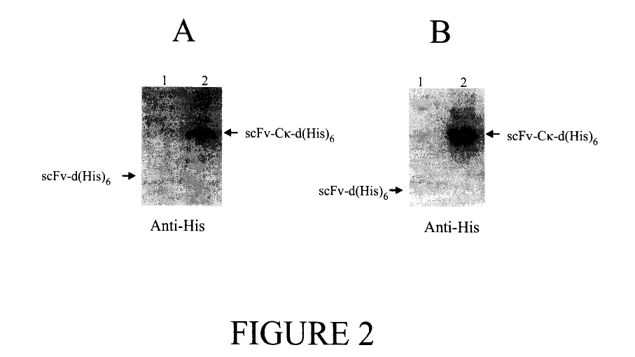

This experiment compared expression of human single chain (sc) antibody fragment constructs with and without the human Cκ domain. The two domain (V_{H}, V_{L}) scFv construct is a standard format for cell based recombinant antibody expression (Holliger, P. & Hudson, P. J. (2005) Nat. Biotechnol. 23, 1126-1136). Anti-carcinoembryonic antigen (CEA) and anti-progesterone scFv fragments, created by eukaryotic ribosome display technology (He, M. & Taussig, M J. (1997) Nucleic Acids Res. 25, 5132-5134) were assembled as fusions to a double (His)₆ tag (d(His)₆) (He, M. & Taussig, M. J. (2001) Nucleic Acid. Res. 29, e73) or additionally to the Cκ domain (Cκ-d(His)₆) by overlap PCR using the constructs as set out in FIG. 1 and using the methods described hereinbefore in sections 2-5. After expression in the E. coli S30 system as described in section 6 above and Western blotting detection by anti-His antibody as described in section 7 showed that neither scFv-d(His)₆ fragment was expressed detectably (lane 1 in FIGS. 2A and 2B), whereas both scFv-Cκ-d(His)₆ fusions successfully led to high expression yields (lane 2 in FIGS. 2A-2B; 2A: anti-progesterone; 2B: anti-CEA).

Comparison with standards estimated that at least 100 μg/ml protein was produced after inclusion of the Cκ domain. Sequencing of the PCR constructs confirmed that the reading frames were in all cases correct and that the only sequence differences were the presence of Cκ. It was also shown that the scFv-Cκ fragments were retained in the soluble fraction after high speed centrifugation (15,000 rpm for 20 min) and bound their specific antigens.

### Example 2

### Effect of Cκ Fusion on GTP and FK506 Binding Protein Expression

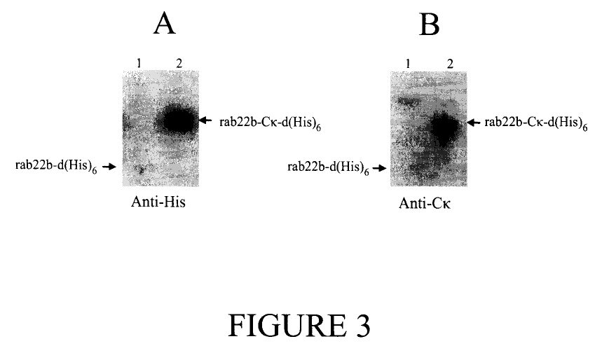

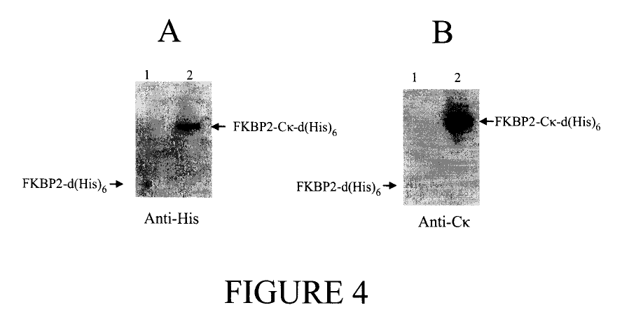

To test whether C-terminal fusion to Cκ could also improve expression of other proteins which were known to be synthesised at very low levels, and particularly non-immunoglobulin related proteins, Rab22b (a GTP binding protein) and FKBP2 (FK506 binding protein) were chosen for this analysis. This experiment was performed in an analogous manner to Example 1 and the results are shown in FIGS. 3-4. Lane 1 of FIGS. 3A and 4A show that both proteins were only poorly expressed in the E. coli S30 lysate as d(His)₆ constructs and were only weakly detected using anti-His antibody. By contrast, the levels of expression of rab22b-Cκ-d(His)₆ and FKBP2-Cκ-d(His)₆ were both strong (lane 2 of FIGS. 3A and 4A). Rab22b-Cκ-d(His)₆ and FKBP2-Cκ-d(His)₆ were also detected using an anti-Cκ monoclonal antibody, confirming high level expression of Cκ fusion (lane 2 of FIGS. 3B and 4B).

When it was possible to detect the non-Cκ tagged protein on a Western blot, the increased expression through inclusion of the Cκ domain was estimated as more than 10-50 fold.

### Example 3

### Effect of Cκ Fusion on Expression of Human Transcription Factors

Materials:

Plasmids encoding the following binding domains of human transcription factors were obtained from National Public Health Institute, Finland:

ERG: Transcriptional regulator ERG

E2F-1: Transcription factor E2F1

SMAD 3: Mothers against decapentaplegic homolog 3

TCF7L2: Transcription factor 7 like 2

Constructs:

(a) Constructs without Cκ Fusion (TF)

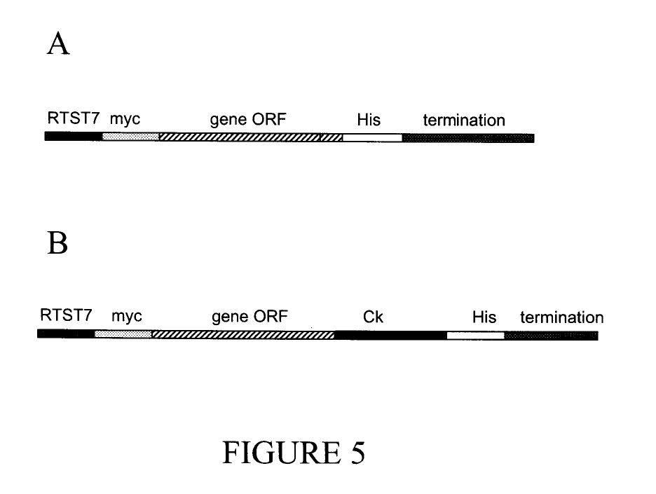

DNA Constructs were generated by PCR. The 5′ end of the PCR constructs contained a T7 promoter, an enhancer and SD sequence followed by ATG. For detection of the expression, both a myc-tag and a double His-tag were added to N-terminus (myc tag) and C-terminus (His-tag) of the target gene. A transcription termination region was also included at the 3′ end of the PCR construct (FIG. 5A)

(b) Constructs with Cκ Fusion (TF-Cκ)

Constructs with Cκ fusion were produced by inserting a Cκ domain between the c-terminus of the target gene and the double His-tag (FIG. 5B).

Results

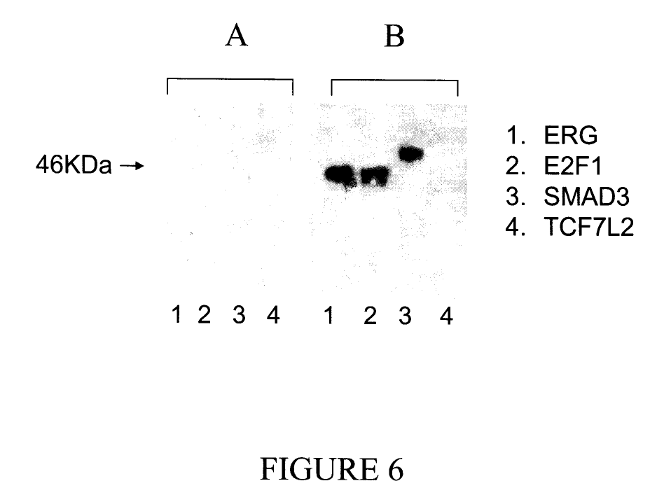

Constructs with or without the Cκ fusion were subjected to protein synthesis in an E. coli cell-free translation kit (Roche). After incubation for 3-4 hrs at 30° C., individual translation mixtures were analysed by SDS-PAGE and Western blot probed by either anti-myc, anti-(His)₆ or anti-Cκ antibodies. The results are shown in FIG. 6 which demonstrates the Western blot result, probed by anti-(His)₆ antibody. FIG. 6 shows that no bands were detected with all four constructs without Cκ fusion (FIG. 6A) and by contrast proteins, which correspond to their respective molecular size, were strongly detected by antibodies in three of the constructs (ERG, E2F-1 and SMAD 3) in the presence of the Cκ domain at the C-terminus (FIG. 6B). The reason for non-detection of the TCF7L2 construct may be due to degradation at mRNA or protein level. It is believed that Cκ expression of the TCF7L2 construct may be achieved by optimisation of expression conditions (such as expression duration and temperature) or addition of RNase or protease inhibitors.

The detection by all the three antibodies (anti-myc, anti-His and anti-Cκ) confirms the expression of these designed epitopes located at both N- and C-terminus of the target gene.

What is claimed is:

1. An in vitro process of expressing a target protein which comprises the steps of:
(a) preparing a nucleic acid construct comprising both a first nucleic acid encoding said target protein and a second nucleic acid encoding an immunoglobulin κ light chain constant domain (Cκ); and
(b) subjecting the construct prepared in step (a) to protein expression in a cell-free protein expression system.

2. The process as defined in claim 1, wherein the cell-free protein expression system comprises components for transcription and translation.

3. The process as defined in claim 1, wherein the cell-free system is a cell-free lysate selected from a prokaryotic or eukaryotic system.

4. The process as defined in claim 1, wherein the cell-free system is an E. coli cell-free system.

5. The process as defined in claim 1, wherein the immunoglobulin κ light chain constant domain (Cκ) is a human immunoglobulin κ light chain constant domain (Cκ).

6. The process as defined in claim 1, wherein the target protein is an immunoglobulin protein.

7. The process as defined in claim 1, wherein the target protein is a non-immunoglobulin protein.

8. The process as defined in claim 1, wherein the target protein is a binding protein.

9. The process as defined in claim 1, wherein the second nucleic acid encoding the Cκ domain is fused to the first nucleic acid encoding the target protein.

10. The process as defined in claim 1, wherein the second nucleic acid encoding the Cκ domain is present at the C-terminus of the first nucleic acid encoding the target protein.

11. The process as defined in claim 1, wherein the nucleic acid construct additionally comprises one or more of the following: a promoter, a transcriptional and translational regulatory sequence, an untranslated leader sequence, a sequence encoding a cleavage site, a recombination site, a transcriptional terminator or a ribosome entry site.

12. The process as defined in claim 1, wherein the nucleic acid construct additionally comprises a third nucleic acid encoding an immobilisation tag configured to attach to a protein immobilisation agent.

13. The process as defined in claim 12, wherein the immobilisation tag is a polyhistidine sequence and said protein immobilisation agent is a chelating agent.

14. The process as defined in claim 13, wherein the immobilisation tag is a peptide, domain or protein and said protein immobilisation agent is an antibody specific to said tag.

15. The process as defined in claim 1, which additionally comprises the step of:
(c) isolating the expressed target protein from the protein expression system.

16. The process as defined in claim 15, wherein the isolation step (c) is performed by affinity chromatography.

17. The process as defined in claim 1, which additionally comprises the step of:
(d) cleaving the Cκ domain from the expressed target protein.

18. The process as defined in claim 1, wherein the protein expression system comprises a biomolecule or a molecule required to produce modifications.

19. A composition for in vitro expression of a target protein, said composition comprising:
(a) a nucleic acid construct comprising both a first nucleic acid encoding said target protein and a second nucleic acid encoding an immunoglobulin κ light chain constant domain (Cκ); and
(b) a cell-free protein expression system.

20. A kit for in vitro protein expression of a target protein which comprises:
(a) a nucleic acid construct comprising a nucleic acid encoding an immunoglobulin κ light chain constant domain (Cκ); and
(b) a cell-free protein expression system.

21. The kit as defined in claim 20, wherein the nucleic acid construct additionally comprises a second nucleic acid encoding said target protein.

22. The kit as defined in claim 20, which additionally comprises instructions to use said kit.


SEQUENCE LISTING (8 sequences)

SEQ ID NO: 1 - DNA, 15 bp, Artificial
  Synthesised Primer
gatctcgatcccgcg

SEQ ID NO: 2 - DNA, 25 bp, Artificial
  Synthesised Primer
catggtggatatctccttcttaaag

SEQ ID NO: 3 - DNA, 17 bp, Artificial
  Synthesised Primer
gctctagaggcggtggc

SEQ ID NO: 4 - DNA, 18 bp, Artificial
  Synthesised Primer
tccggatatagttcctcc

SEQ ID NO: 5 - DNA, 19 bp, Artificial
  Synthesised Primer
gtggctgcaccatctgtct

SEQ ID NO: 6 - DNA, 37 bp, Artificial
  Synthesised Primer
agatggtgcagccacagttttgtacaagaaagctggg

SEQ ID NO: 7 - DNA, 45 bp, Artificial
  Synthesised Primer
cttaagaaggagatatccaccatgctcgaatcaacaagtttgtac

SEQ ID NO: 8 - DNA, 36 bp, Artificial
  Synthesised Primer
gccaccgcctctagagcgtttgtacaagaaagctgg